(19) United States
(12) Patent Application Publication
(10) Pub. No.: US 2021/0240433 A1
(43) Pub. Date: Aug. 5, 2021

(54) INFORMATION PROCESSING APPARATUS AND NON-TRANSITORY COMPUTER READABLE MEDIUM

(71) Applicant: FUJI XEROX CO., LTD., Tokyo (JP)

(72) Inventors: Masahiro SATO, Kanagawa (JP); Kengo TOKUCHI, Kanagawa (JP)

(73) Assignee: FUJI XEROX CO., LTD., Tokyo (JP)

(21) Appl. No.: 16/987,746

(22) Filed: Aug. 7, 2020

(30) Foreign Application Priority Data
Feb. 4, 2020 (JP) 2020-017479

(51) Int. Cl. G06F 3/16; G10L 21/043; G10L 25/51; G06F 3/01

(52) U.S. Cl. CPC G06F 3/165; G06F 3/015; G10L 25/51; G10L 21/043

(57) ABSTRACT

An information processing apparatus includes a processor configured to, if information regarding a psychological state or a feeling of a target satisfies a predetermined first condition, control outputting of sound around the target collected in a period in which the predetermined first condition is satisfied.

### CROSS-REFERENCE TO RELATED APPLICATIONS

This application is based on and claims priority under 35 USC 119 from Japanese Patent Application No. 2020-017479 filed Feb. 4, 2020.

### BACKGROUND

### (i) Technical Field

The present disclosure relates to an information processing apparatus and a non-transitory computer readable medium.

### (ii) Related Art

Use of biological information such as brain waves is expected as a next-generation user interface. For example, there is an image capture control apparatus including a condition storage unit that stores in advance a biological information condition, which is a condition of biological information caused when a user imagines a certain physical action, a condition determination unit that obtains biological information relating to information from a living body and that determines whether information included in the obtained biological information satisfies the biological information condition stored in the condition storage unit, and a condition output unit that, if the condition determination unit determines that the biological information condition is satisfied, outputs an image capture condition, which is a condition for an image capture apparatus to capture an image of a subject, to the image capture apparatus (e.g., refer to Japanese Unexamined Patent Application Publication No. 2015-229040).

### SUMMARY

When a person is talking with others or attending a meeting, the person might miss what the others say. Such a situation tends to occur when the person is not concentrating or excited.

Aspects of non-limiting embodiments of the present disclosure relate to provision of an additional opportunity to check surrounding sound if information regarding a psychological state or a feeling of a target satisfies a predetermined condition.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including a processor configured to, if information regarding a psychological state or a feeling of a target satisfies a predetermined first condition, control outputting of sound around the target collected in a period in which the predetermined first condition is satisfied.

### BRIEF DESCRIPTION OF THE DRAWINGS

Exemplary embodiments of the present disclosure will be described in detail based on the following figures, wherein:

FIGS. 1A and 1B are diagrams illustrating an example of an earphone terminal worn by a person: FIG. 1A illustrates the earphone terminal worn by the person viewed diagonally from the front, and FIG. 1B illustrates the earphone terminal worn by the person viewed from the front.

FIGS. 2A and 2B are diagrams illustrating an example of the appearance of the earphone terminal used in a first exemplary embodiment: FIG. 2A illustrates the appearance of the entirety of the earphone terminal, and FIG. 2B illustrates the appearance of left and right modules;

FIG. 3 is a diagram illustrating an example of the internal configuration of the earphone terminal;

FIG. 4 is a diagram illustrating an example of the functional configuration of the earphone terminal;

FIG. 5 is a flowchart illustrating an example of a process performed by the earphone terminal used in the exemplary embodiment;

FIG. 6 is a diagram illustrating an example in which sound is played back when an irritated person has calmed down;

FIG. 7 is a diagram illustrating an example in which the wearer gives, after calming down, an instruction to play back sounds uttered during a meeting;

FIG. 8 is a diagram illustrating a measurement point of a headset equipped with a brain wave sensor capable of measuring brain waves with the earphone terminal worn a user;

FIG. 9 is a diagram illustrating measurement points for brain waves described in a thesis;

FIG. 10 is a diagram illustrating evaluation of output a waves;

FIGS. 11A and 11B are diagrams illustrating results of measurement performed with MindWave: FIG. 11A illustrates a result of measurement at a time when an open eye state and a closed eye state are alternated twice with subjects whose blinking is vague, and FIG. 11B illustrates a result of measurement at a time when the open eye state and the closed eye state are alternated twice with subjects whose blinking is clear;

FIGS. 12A and 12B are diagrams illustrating results of measurement performed with the earphone terminal used in the exemplary embodiment: FIG. 12A illustrates a result of measurement at a time when the open eye state and the closed eye state are alternated twice with subjects whose blinking is vague, and FIG. 12B illustrates a result of measurement at a time when the open eye state and the closed eye state are alternated twice with subjects whose blinking is clear and who are asked to move the jaw;

FIGS. 13A to 13C are diagrams illustrating other results of the measurement performed with MindWave: FIG. 13A illustrates changes in the percentage of spectral intensity in each frequency band at a time when subjects have entered the closed eye state from the open eye state with clear blinking, FIG. 13B illustrates changes in the percentage of spectral intensity in each frequency band at a time when the subjects have entered the closed eye state from the open eye state with vague blinking, and FIG. 13C illustrates a case where a waves do not increase;

FIGS. 14A to 14C are diagrams illustrating other results of the measurement performed with the earphone terminal used in the exemplary embodiment: FIG. 14A illustrates changes in the percentage of spectral intensity in each frequency band at a time when the subjects have entered the closed eye state from the open eye state with clear blinking, FIG. 14B illustrates changes in the percentage of spectral intensity in each frequency band at a time when the subjects have entered the closed eye state from the open eye state with vague blinking, FIG. 14C illustrates a case where a waves do not increase;

FIGS. 15A and 15B are diagrams illustrating an example of presentation of parts in which spectral intensity has increased: FIG. 15A illustrates a result of the measurement performed with MindWave, and FIG. 15B illustrates a result of the measurement performed with the earphone terminal used in the exemplary embodiment;

FIG. 16 is a flowchart illustrating an example of a process performed by an earphone terminal used in a second exemplary embodiment;

FIG. 17 is a diagram illustrating a case where an external apparatus is a server on the Internet;

FIG. 18 is a diagram illustrating an example of the appearance of an earphone terminal to be inserted into one of the ears;

FIG. 19 is a diagram illustrating an example of an earring for which electrodes for measuring brain waves are provided;

FIG. 20 is a diagram illustrating an example of spectacles for which the electrodes for measuring brain waves are provided;

FIGS. 21A and 21B are diagrams illustrating an example in which the electrodes for measuring brain waves are provided for a headset having a function of displaying an image assimilating to a surrounding environment of the user;

FIG. 22 is a diagram illustrating an example of a headset that measures changes in the amount of blood flow caused by brain activity using near-infrared light; and

FIG. 23 is a diagram illustrating an example of a magnetoencephalograph (MEG).

### DETAILED DESCRIPTION

Exemplary embodiments of the present disclosure will be described hereinafter with reference to the drawings.

### First Exemplary Embodiment

### System Configuration

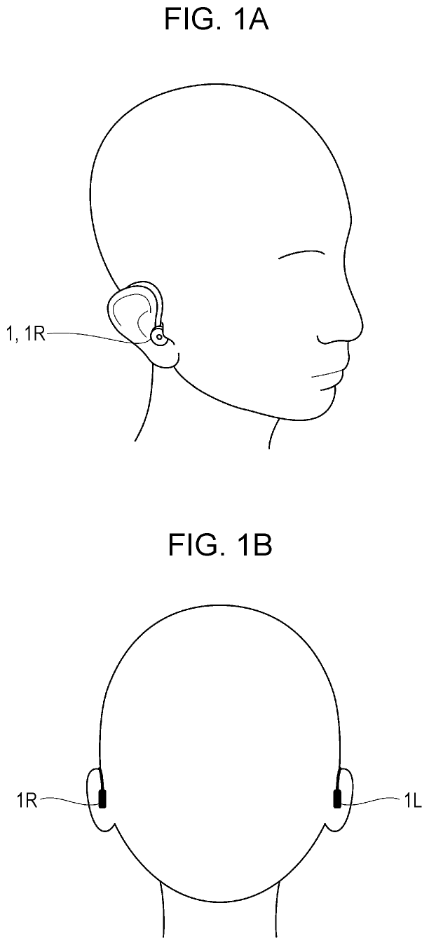

FIGS. 1A and 1B are diagrams illustrating an example of an earphone terminal 1 worn by a person. FIG. 1A illustrates the earphone terminal 1 worn by the person (hereinafter referred to as a “wearer”) viewed diagonally from the front, and FIG. 1B illustrates the earphone terminal 1 worn by the wearer viewed from the front.

The earphone terminal 1 according to the present exemplary embodiment is an example of an information processing apparatus and includes a module 1R attached to the right ear and a module 1L attached to the left ear.

The wearer in the present exemplary embodiment is an example of a target.

The earphone terminal 1 according to the present exemplary embodiment includes a circuit that plays back sounds received from an audio device or a smartphone, which is not illustrated, and a circuit that measures electrical signals caused by brain activity (hereinafter referred to as “brain waves”).

The earphone terminal 1 used in the present exemplary embodiment is a wireless device. The earphone terminal 1, therefore, is connected to an external apparatus through wireless communication. The external apparatus herein may be an audio player, a smartphone, a tablet terminal, a laptop computer, or a wearable computer. Bluetooth (registered trademark), for example, is used for the communication with the external apparatus. Alternatively, another communication standard such as Wi-Fi (registered trademark) may be used for the wireless communication. Alternatively, the earphone terminal 1 and the external apparatus may be connected to each other by cable.

The earphone terminal 1 is used to measure brain waves in order to take into consideration the spread of interfaces employing brain waves.

When interfaces employing brain waves will begin to spread in the future, users might not like wearing devices visibly designed to measure brain waves. Helmet-shaped devices, for example, might not gain popularity in terms of both design and a burden on the body.

For this reason, the earphone terminal 1 will be focused upon in the present exemplary embodiment as a device for measuring brain waves. Since earphones are already widely used as an audio device, users will not be reluctant to wear the earphone terminal 1 in terms of appearance.

In addition, since the external auditory meatuses, into which the earphone terminal 1 is inserted, are close to the brain, brain waves can be easily measured. How the earphone terminal 1 can measure brain waves will be described later in a “Results of Experiments” section. The external auditory meatuses are an example of the ears. In the present exemplary embodiment, the ears include the auricles and the external auditory meatuses.

The earphone terminal 1 also includes cartilage conduction vibrators. Sound conduction achieved by the cartilage conduction vibrators is called “cartilage conduction”. In cartilage conduction, the external auditory meatuses need not be blocked. The wearer, therefore, can hear cartilage conduction sound and external sound at the same time.

A pathway used by cartilage conduction is called a “third auditory pathway”, which is different from an air conduction pathway and a bone conduction pathway.

The earphone terminal 1 according to the present exemplary embodiment includes both a circuit for measuring the wearer's brain waves and a circuit for transmitting sound to the wearer through cartilage conduction.

### Configuration of Earphone Terminal 1

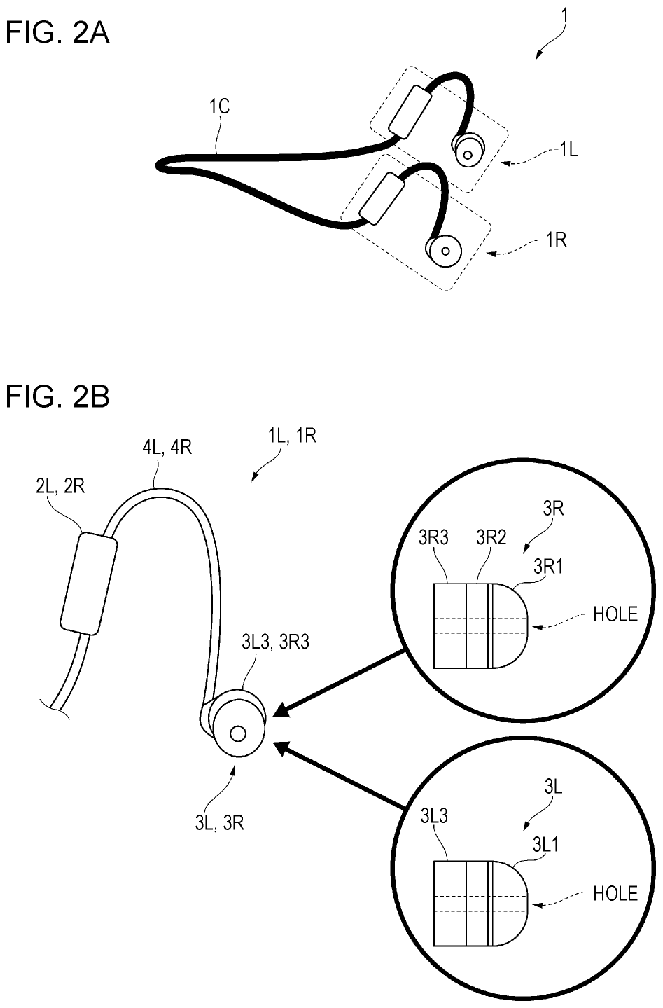

FIGS. 2A and 2B are diagrams illustrating an example of the appearance of the earphone terminal 1 used in the first exemplary embodiment. FIG. 2A illustrates the appearance of the entirety of the earphone terminal 1, and FIG. 2B illustrates the appearance of the left and right modules 1L and 1R.

The earphone terminal 1 according to the present exemplary embodiment includes the module 1L attached to the left ear, the module 1R attached to the right ear, and a connection 1C that connects the modules 1L and 1R to each other. The connection 1C is composed of a resin and includes a power line and a signal line.

The module 1L attached to the left ear includes a module body 2L storing a battery and the like, a vibration unit 3L that is provided with an electrode and that is attached to the ear, and an ear hook 4L attached to a gap between the auricle and the temple.

Similarly, the module 1R attached to the right ear includes a module body 2R storing an electronic circuit and the like, a vibration unit 3R that is provided with electrodes and that is attached to the ear, and an ear hook 4R attached to a gap between the auricle and the temple.

The vibration units 3L and 3R provided with the electrodes according to the present exemplary embodiment include ring-shaped electrode units that come into contact with the inner walls of the external auditory meatuses and cartilage conduction vibrators 3L3 and 3R3, respectively, that come into contact with the auricles.

The electrode unit for the left module 1L includes a dome-shaped electrode 3L1 having a through-hole at the center thereof. The electrode unit for the right module 1R includes a dome-shared electrode 3R1 having a through-hole at the center thereof and a ring-shared electrode 3R2 that comes into contact with the concha cavity.

The cartilage conduction vibrators 3L3 and 3R3 are elements that generate vibration necessary for cartilage conduction. The cartilage conduction vibrators 3L3 and 3R3 according to the present exemplary embodiment are covered by protection members. That is, the cartilage conduction vibrators 3L3 and 3R3 are closed-type vibrators.

In the present exemplary embodiments, the vibration units 3L and 3R provided with the electrodes each have a hole extending from a deep part of the ear to the outside. The wearer of the vibration units 3L and 3R, therefore, can hear external sound through air conduction pathways.

The electrodes 3L1, 3R1, and 3R2 according to the present exemplary embodiment are composed of conductive rubber in order to measure electrical signals on the skin. The electrodes 3R1 and 3R2 are electrically isolated from each other by an insulator.

In the present exemplary embodiment, the electrode 3R1 is a terminal used to obtain an electroencephalogram (EEG) (hereinafter referred to as an “EEG measuring terminal”). Potential variations measured by the electrode 3R1 include potential variations caused by not only brain waves but also other types of biological information. The electrode 3R2 is a ground (GND) electrode.

The electrode 3L1, on the other hand, is a terminal used to measure a reference (REF) potential (hereinafter referred to as a “REF terminal”). In the present exemplary embodiment, the electrodes 3R2 and 3L1 are electrically isolated from each other by an insulator.

In the present exemplary embodiment, potential variations caused by brain waves are measured as differential signals between electrical signals measured by the electrodes 3R1 and 3L1. The same holds for potential variations caused by the other types of biological information.

Brain waves and biological information other than the brain waves will be generically referred to as “biological information such as brain waves”.

In the field of brain science, all potential variations derived from biological information other than brain waves are called “artifacts”. It is considered that electrical signals obtained by measuring brain waves invariably include artifacts.

Components of artifacts are classified into those derived from a living body, those derived from a measurement system including electrodes, and those derived from external devices and environment. Among these three types of component, the components other than those derived from a living body can be detected by the earphones 10 as noise. The noise can be measured as electrical signals at a time when the electrodes 3R1 and 3L1 are electrically short-circuited to each other.

The module 1R according to the present exemplary embodiment includes a circuit that measures the wearer's brain waves and the like, a circuit that analyzes the measured brain waves and that identifies information regarding a psychological state or a feeling (hereinafter referred to as a “psychological state or the like”), and a circuit that controls recording and playback of sound around the wearer in accordance with the psychological state or the like of the wearer. The module 1L, on the other hand, includes a battery.

In the present exemplary embodiment, information regarding a psychological state or the like is not limited to verbal information but may be information represented by codes, signs, numerical values, or the like, instead.

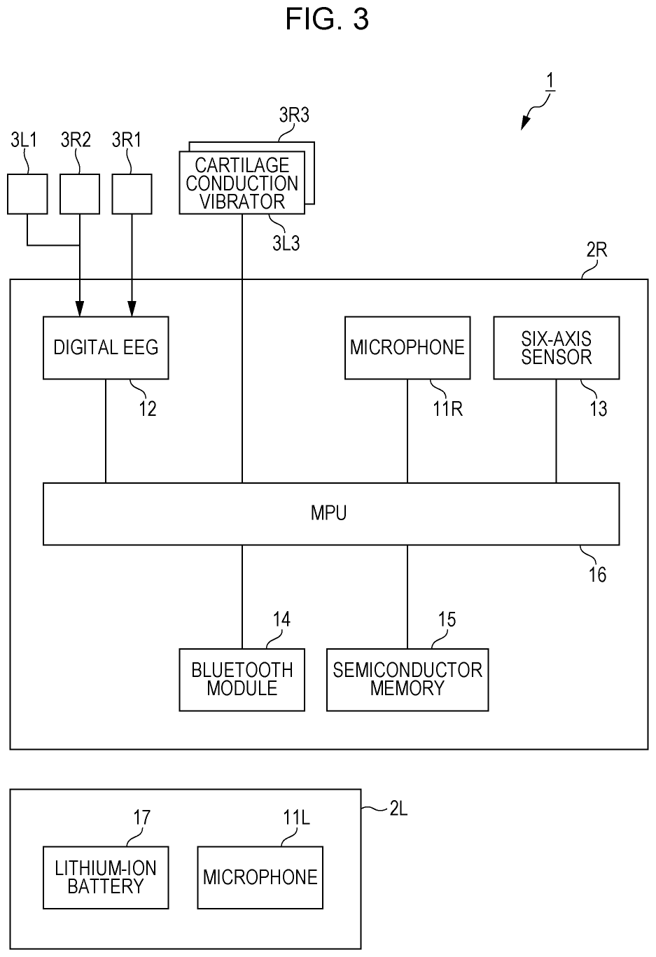

FIG. 3 is a diagram illustrating an example of the internal configuration of the earphone terminal 1.

The module body 2R includes a microphone 11R, a digital electroencephalograph (EEG) 12, a six-axis sensor 13, a Bluetooth module 14, a semiconductor memory 15, and a microprocessor unit (MPU) 16.

The digital EEG 12 includes a differential amplifier that differentially amplifies potential variations detected by the electrodes 3R1 and 3L1, a sampling circuit that samples outputs of the differential amplifier, and an analog-to-digital (A/D) conversion circuit that converts an analog potential after the sampling into a digital value. In the present exemplary embodiment, a sampling rate is 600 Hz. The resolution of the A/D conversion circuit is 16 bits.

The six-axis sensor 13 includes a three-axis acceleration sensor and a three-axis gyro sensor. The six-axis sensor 13 is used to detect an attitude of the user.

The Bluetooth module 14 communicates data with the external apparatus, which is not illustrated. The Bluetooth module 14 is used, for example, to receive audio data from the external apparatus.

The semiconductor memory 15 includes, for example, a read-only memory (ROM) storing basic input-output system (BIOS), a random-access memory (RAM) used as a working area, and a rewritable nonvolatile memory (hereinafter referred to as a “flash memory”).

In the present exemplary embodiment, the flash memory is used to record sounds collected by the microphone 11R, digital signals that are outputs of the digital EEG 12, information regarding a psychological state or the like identified as a result of an analysis of brain waves, and audio data received from the external apparatus. The flash memory also stores firmware and application programs.

The MPU 16 analyzes brain waves measured by the digital EEG 12 and controls playback of surrounding sound in accordance with a psychological state or the like obtained as a result of the analysis. When analyzing brain waves, the MPU 16 performs processing, such as a Fourier transform, on digital signals output from the digital EEG 12. The MPU 16 and the semiconductor memory 15 operate as a computer.

The module body 2L, on the other hand, includes a microphone 11L and a lithium-ion battery 17.

### Functional Configuration of Earphone Terminal 1

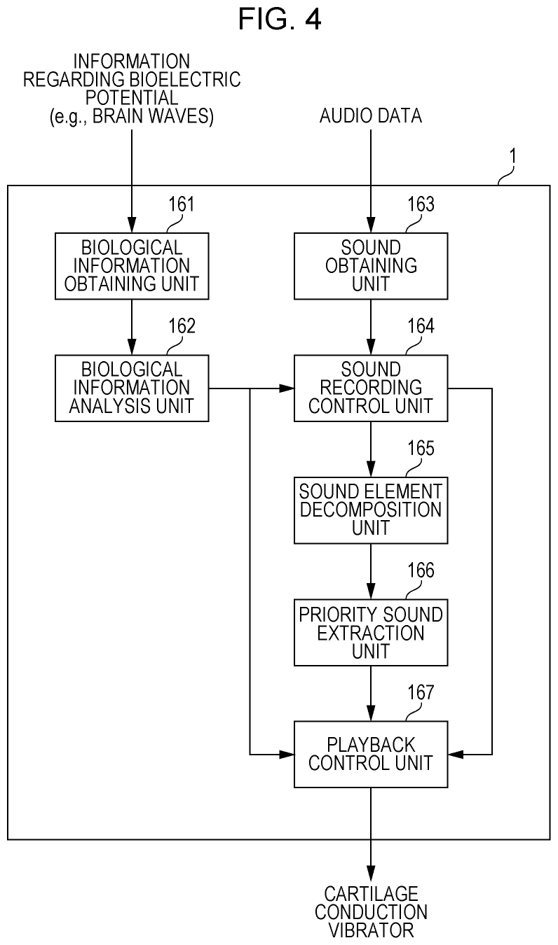

FIG. 4 is a diagram illustrating an example of the functional configuration of the earphone terminal 1. The functions illustrated in FIG. 4 are achieved by cooperation between the MPU 16 (refer to FIG. 3) and various components.

The earphone terminal 1 according to the present exemplary embodiment functions as a biological information obtaining unit 161 that obtains biological information including brain wave information from information regarding bioelectric potentials, a biological information analysis unit 162 that analyzes obtained biological information and that estimates the psychological state or the like of the wearer, a sound obtaining unit 163 that obtains data (hereinafter referred to as “audio data”) regarding sound around the wearer output from the microphones 11L and 11R, a sound recording control unit 164 that controls recording of obtained audio data in accordance with the biological information and the like, a sound element decomposition unit 165 that decomposes recorded audio data into sound elements, a priority sound extraction unit 166 that extracts sound elements in a predetermined order of priority, and a playback control unit 167 that controls playback of audio data on the basis of predetermined conditions.

The biological information obtaining unit 161 and the biological information analysis unit 162 may be achieved as functions of the digital EEG 12 (refer to FIG. 3) or functions of the MPU 16 (refer to FIG. 3).

The biological information obtaining unit 161 according to the present exemplary embodiment obtains features of brain waves from information regarding bioelectric potentials. The biological information analysis unit 162 according to the present exemplary embodiment uses an independent component analysis (ICA) or another known technique to obtain features of brain waves. The features of brain waves include, for example, waveform components unique to brain waves, the spectral intensity and distribution of each frequency component included in the waveform components, the spectral intensity of certain frequency components included in the waveform component, and the percentage of increase in a waves.

In the present exemplary embodiment, the biological information analysis unit 162 conducts a frequency analysis on brain waves through a fast Fourier transform or the like to generate an n×m data matrix, whose rows represent time and whose columns represent frequency components. The biological information analysis unit 162 then normalizes the n×m data matrix and obtains a correlation matrix from the normalized data matrix. Next, the biological information analysis unit 162 decomposes the correlation matrix into eigenvectors and extracts factors through a principal factor analysis. Next, the biological information analysis unit 162 performs a varimax rotation using extracted factors whose contribution rates are high, obtains factor scores using a method of least squares, and determines the obtained factor scores as feature values. In the present exemplary embodiment, feature values obtained in this manner are used as biological information indicating the psychological state or the like of the wearer of the earphone terminal 1. A method for obtaining feature values is not limited to this, and another method may be used, instead.

The biological information analysis unit 162 according to the present exemplary embodiment classifies biological information into plural psychological states and the like. In the present exemplary embodiment, the plural psychological states or the like are, for example, “like”, “dislike”, “pleasant”, “sad”, “dangerous”, “interested”, “sleepy”, “concentrating”, “relaxed”, “sharp”, “stressed”, “angry”, “excited”, and “happy”. These are just examples, and more or fewer psychological states or the like may be used, instead. These are an example of verbal classifications.

The sound obtaining unit 163 obtains audio data output from the microphones 11L and 11R (refer to FIG. 3) and converts the audio data into a predetermined data format.

The sound recording control unit 164 according to the present exemplary embodiment records, in the semiconductor memory 15 (refer to FIG. 3), audio data obtained in periods in which information relating to biological information and the like has satisfied at least one of the predetermined conditions and does not record audio data obtained in periods in which the information has not satisfied none of the predetermined conditions. Alternatively, all audio data may be recorded in the semiconductor memory 15 regardless of the information relating to the biological information and the like. The predetermined conditions are examples of a first condition.

The sound recording control unit 164 according to the present exemplary embodiment records obtained audio data in the semiconductor memory 15 when it is determined that the wearer is not concentrating.

A state in which it is determined that the wearer is not concentrating may be, for example, a case where it is determined that the wearer is excessively relaxed, a case where it is determined that the wearer is sleepy or asleep, or a case where it is determined that the wearer is bored. In these cases, the wearer might not be able to fully understand what others are talking about.

When the wearer is not concentrating, a level of θ waves measured in the wearer might be higher than a predetermined threshold or a level of δ waves measured in the wearer might be higher than a predetermined threshold. θ waves are a frequency component whose frequency is within a range of about 4 Hz to about 8 Hz, and δ waves are a frequency component whose frequency is about 4 Hz or lower.

The sound recording control unit 164, therefore, may be provided with a function of detecting a state in which the wearer is not concentrating as a case where the level of θ waves is higher than the predetermined threshold or a case where the level of δ waves is higher than the predetermined threshold. The state in which the wearer is not concentrating is an example of the first condition.

When it is determined that the wearer is aroused, the sound recording control unit 164 according to the present exemplary embodiment records obtained audio data in the semiconductor memory 15.

A state in which the wearer is aroused may be, for example, a case where it is determined that the wearer is irritated or a case where it is determined that the wearer is excited or excessively excited. In these cases, too, the wearer might not be able to fully understand what others are talking about.

When the wearer is aroused, a level of γ waves measured in the wearer might be higher than a predetermined threshold or a level of β waves measured in the wearer might be higher than a predetermined threshold. γ waves are a frequency component whose frequency is within a range of about 40 Hz to about 70 Hz, and β waves are a frequency component whose frequency is within a range of about 13 Hz to about 40 Hz.

The sound recording control unit 164, therefore, may be provided with a function of detecting a state in which the wearer is aroused as a case where the level of γ waves is higher than the predetermined threshold or a case where the level of β waves is higher than the predetermined threshold. The state in which the wearer is aroused is an example of the first condition. The first condition may be set for each account.

The sound element decomposition unit 165 performs a process for decomposing audio data recorded in the semiconductor memory 15 (refer to FIG. 3) into sound elements. In the present exemplary embodiment, the sound element decomposition unit 165 decomposes audio data into sound elements using plural criteria. The criteria include, for example, sound types, differences in a sound source or a speaker, a word unit, and a summary.

When the sound types are used as a criterion, audio data is decomposed into, for example, human voices and other sounds. Alternatively, audio data may be decomposed into other types. The number of types may be three or more, instead.

When the differences in a sound source or a speaker are used as a criterion, audio data is decomposed, for example, in accordance with speakers. For example, audio data is decomposed into A's voice and B's voice. A technique for recognizing speakers from audio data has already been put into practice. The technique may be, for example, one of Speaker Recognition application programming interfaces (APIs) developed by Microsoft.

When the word unit is used as a criterion, audio data is decomposed, for example, in units of phrases or words. When audio data is decomposed in units of phrases or words, phrases or words that frequently appear can be extracted.

When the summary is used as a criterion, a summary is generated from audio data using a known technique. For example, there is a technique for converting audio data into text data and generating a summary of the text data. When a summary is generated, a summary of a talk can also be extracted.

The priority sound extraction unit 166 performs a process for extracting sound elements in accordance with a predetermined order of priority. The wearer sets the order of priority. In the present exemplary embodiment, the order of priority is set in advance. The order of priority defines a relationship between priority levels of sound elements to be played back. Sound elements to be played back are determined on the basis of the order of priority.

An example of sound elements having a high priority level is sound elements corresponding to certain speakers. A typical example of the certain speakers is bosses and leaders. More specifically, priority levels of the certain speakers are set high.

Another example of the sound elements having a high priority level is sound elements corresponding to certain speakers who speak a lot. These certain speakers, too, might be bosses and leaders, but speakers who speak a lot are likely to make remarks to be noted.

Another example of the sound elements having a high priority level is phrases and words that frequently appear. By giving priority to phrases and words that frequently appear, the gist of a talk can be recognized in a short period of time.

Another example of the sound elements having a high priority level is a summary of a talk. When a summary is played back, the gist of a talk can be recognized in a short period of time.

The order of priority need not necessarily be set. In this case, all of recorded audio data is played back. The order of priority is an example of a third condition. The order of priority may be set for each wearer. In other words, the order of priority may be set for each account.

If one of the predetermined conditions is satisfied, the playback control unit 167 plays back audio data or sound elements recorded in the semiconductor memory 15 (refer to FIG. 3).

One of the predetermined conditions is, for example, that a state relating to the psychological state or the like of the wearer is a state in which the wearer can understand a talk. In other words, the predetermined condition is that the wearer no longer lacks concentration or is no longer aroused. That is, the predetermined condition is that the wearer has regained concentration or calmed down. The predetermined condition can be defined as a case where the first condition is not satisfied.

Another predetermined condition may be that the wearer has given an explicit instruction. The explicit instruction is input through an operation performed on an operator or an operation button, which is not illustrated. In this case, the wearer can select a timing at which audio data is to be played back. In other words, the wearer can play back recorded audio data at a convenient timing.

Another predetermined condition may be that a change in an environment of the wearer is detected. A change in the environment is, for example, an end of a talk or an end of a meeting. An end of a speech is identified, for example, by detecting a word for ending a talk. An end of a meeting is identified, for example, by detecting a word for ending a meeting or an increase in noise.

Another predetermined condition may be real-time playback. In this case, collected sounds are played back even when it is difficult for the wearer to understand a talk. In many cases, the wearer hears a sound transmitted through cartilage conduction louder than a sound that directly enters the ears. Even when the psychological state or the like of the wearer indicates that it is difficult for the wearer to recognize surrounding sound, the wearer can pay attention to the collected sounds. Since the earphone terminal 1 according to the present exemplary embodiment is not a hearing aid, audio data and the like are played back in real-time only when the psychological state or the like of the wearer indicates that it is difficult for the wearer to understand a talk.

A predetermined condition employed by the playback control unit 167 is an example of a second condition. The second condition, too, is set for each wearer. In other words, the second condition may be set for each account.

### Process

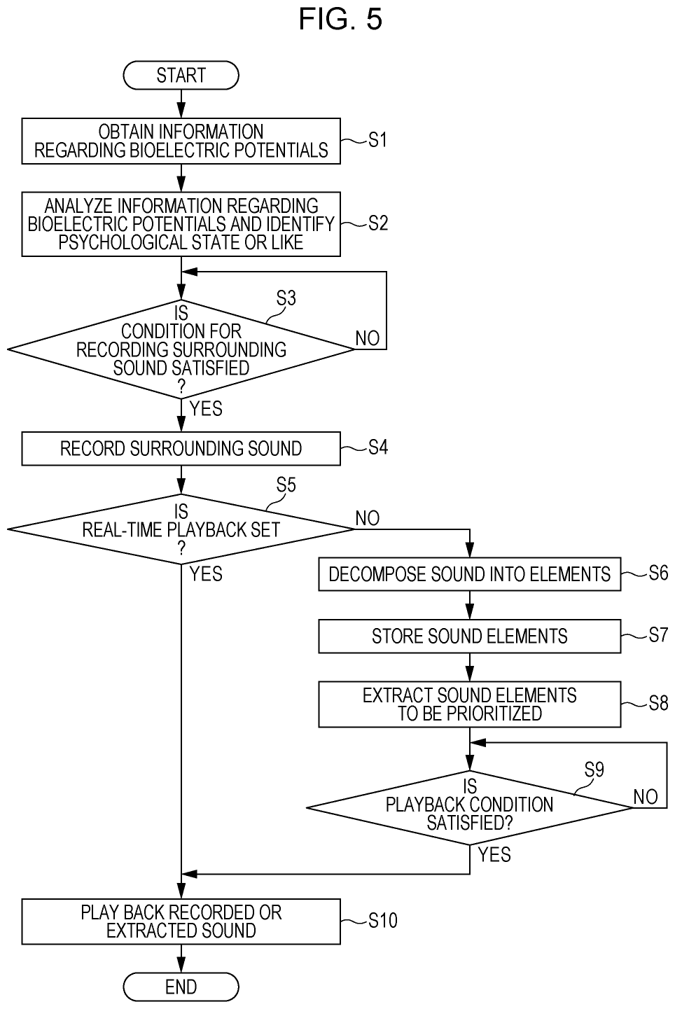

FIG. 5 is a flowchart illustrating an example of a process performed by the earphone terminal 1 used in the first exemplary embodiment. In FIG. 5, steps of the process are indicated by “S”.

First, the earphone terminal 1 obtains information regarding bioelectric potentials (S1). The earphone terminal 1 then analyzes the information regarding bioelectric potentials and identifies a psychological state or the like (S2). In the present exemplary embodiment, the information regarding bioelectric potentials is information including brain waves, and one or more of prepared psychological states and the like are identified.

Next, the earphone terminal 1 determines whether a condition for recording surrounding sound is satisfied (S3). While a result of S3 is negative, the earphone terminal 1 repeats S3. In this period, the surrounding sound of the wearer is not recorded. The surrounding sound is not transmitted through cartilage conduction, either.

If the result of S3 is positive, the earphone terminal 1 records the surrounding sound (S4).

Next, the earphone terminal 1 determines whether real-time playback is set (S5).

If a result of S5 is positive, the earphone terminal 1 plays back the recorded or extracted sound (S10). Here, the recorded sound is played back in real-time. The cartilage conduction vibrators 3L3 and 3R3 (refer to FIG. 3) are used for the playback.

If the result of S5 is negative, on the other hand, the earphone terminal 1 decomposes the sound into sound elements (S6) and stores the obtained sound elements (S7). As described above, the sound elements are stored in the semiconductor memory 15 (refer to FIG. 3).

Next, the earphone terminal 1 extracts sound elements to be prioritized (S8). The earphone terminal 1 extracts the sound elements on the basis of a predetermined order of priority.

The earphone terminal 1 then determines whether a playback condition is satisfied (S9).

While the playback condition is not satisfied, the earphone terminal 1 obtains a negative result in S9. If the playback condition is satisfied, the earphone terminal 1 obtains a positive result in S9. The process proceeds to S10, and the earphone terminal 1 plays back the extracted sound elements.

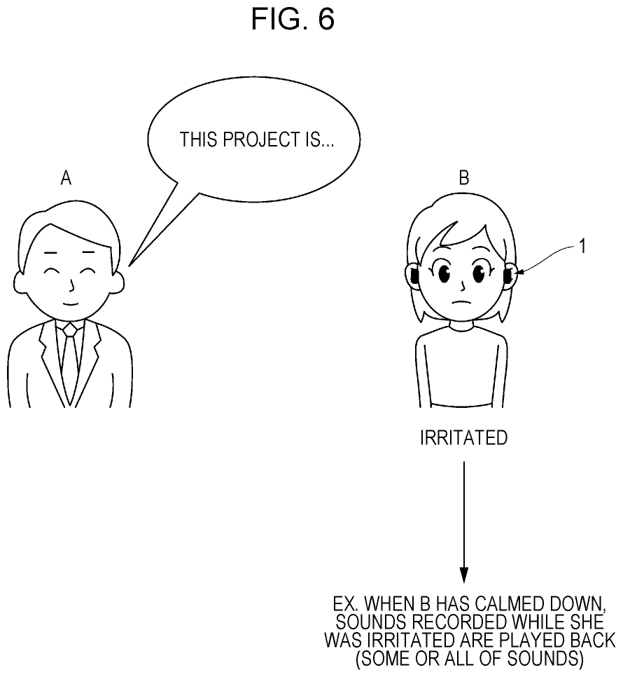

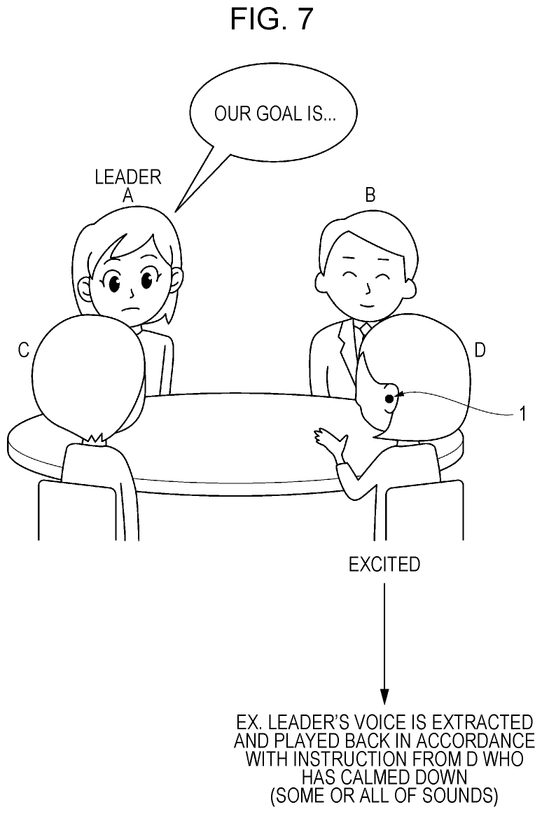

An example of use of the earphone terminal 1 will be described with reference to FIGS. 6 and 7.

FIG. 6 is a diagram illustrating an example in which sound is played back when an irritated person has calmed down. In FIG. 6, A is a speaker, and B, who wears the earphone terminal 1, is a listener. As illustrated in FIG. 6, A is saying, “This project is . . . ”, to B, but B is irritated and does not fully understand what A is talking about. Since the earphone terminal 1 is provided with air conduction pathways, B can physically hear A's voice. B, however, is irritated and not in a suitable state for understanding a talk.

In this case, when the earphone terminal 1 detects that B has calmed down, the earphone terminal 1 starts to play back sounds recorded while B was irritated. Sounds to be played back change depending on predetermined settings. For example, the entirety of the sounds is played back at normal speed or faster than normal speed. Alternatively, for example, a summary of a speech is selectively played back.

FIG. 7 is a diagram illustrating an example in which the wearer gives, after calming down, an instruction to play back sounds uttered during a meeting. In FIG. 7, A, B, C, and D attend the meeting. In FIG. 7, A is a leader and saying, “Our goal is . . . ”. At this time, B, C, and D are listeners. D wears the earphone terminal 1. Possibly because of nervousness, D is excited. D, therefore, does not fully understand what A is talking about.

In the example illustrated in FIG. 7, D, after calming down, gives an instruction to extract the leader's voice and start to play back the leader's voice. In FIG. 7, D has set a priority level of A's voice high. Even if B or C has spoken, A's voice is selectively played back. Since A's voice is played back at the request of D in this example, D can check, even during the meeting, what A has talked about without being noticed by the other attendees.

### Results of Experiments

A fact that the earphone terminal 1 (refer to FIGS. 2A and 2B) can obtain the wearer's brain waves will be described hereinafter on the basis of results of an experiment conducted by a third party and results of an experiment conducted by the present applicant.

### Reliability of MindWave (NeuroSky) in Comparison with Earphone Terminal 1

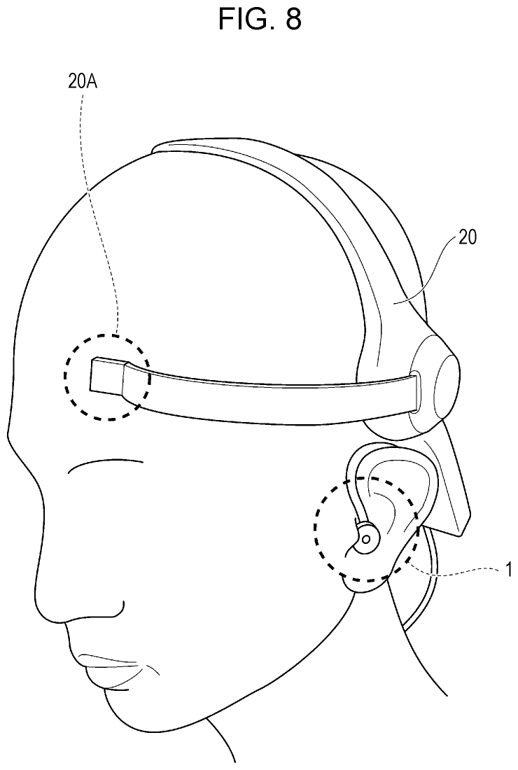

FIG. 8 is a diagram illustrating a measurement point of a headset 20 equipped with a brain wave sensor capable of measuring brain waves with the earphone terminal 1 worn by a user.

In this experiment, MindWave (registered trademark), which is manufactured by NeuroSky, Inc. and commercially available, is used as the headset 20 equipped with a brain wave sensor.

Whereas the earphone terminal 1 uses the external auditory meatuses as measurement points for brain waves as described above, MindWave manufactured by NeuroSky Inc. uses a forehead 20A as a measurement point for brain waves.

The forehead 20A illustrated in FIG. 8 corresponds to Fp1, which is one of 21 sites specified in the 10-20 system recommended as an international standard of arrangement of electrodes for measuring brain waves.

Elena Ratti, et al., “Comparison of Medical and Consumer Wireless EEG Systems for Use in Clinical Trials” (https://www.frontiersin.org/articles/10.3389/fnhum.2017.003 98/full) has verified that brain waves measured by MindWave are equivalent to ones measured by medically approved EEG systems.

This thesis has been reviewed by Dimiter Dimitrov, PhD, a senior scientist at Duke University, and Marta Parazzini, PhD, at Polytechnic University of Milan and the National Research Council (CNR) in Italy.

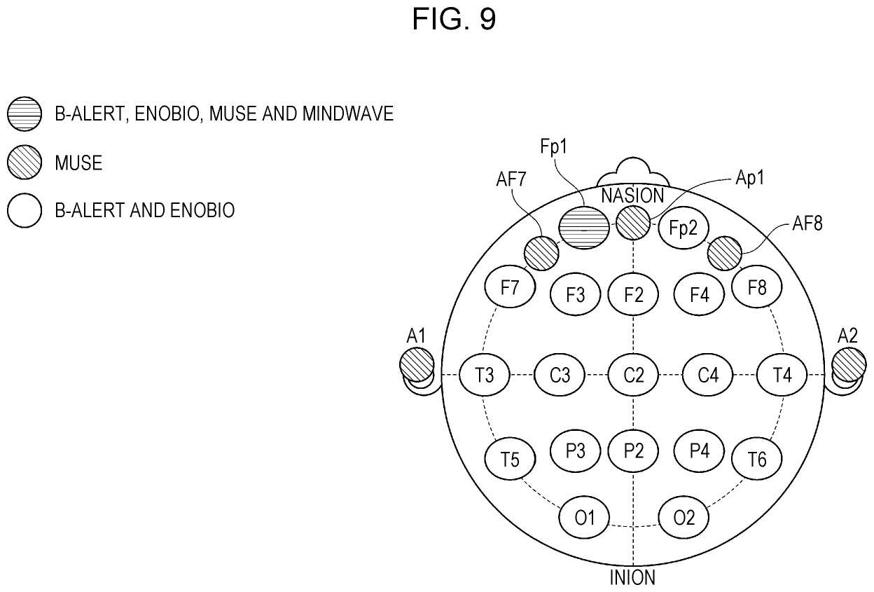

FIG. 9 is a diagram illustrating measurement points for brain waves used in the thesis.

“B-Alert” (registered trademark) and “Enobio” in FIG. 9 are names of medically approved EEG systems in Europe and the U.S. “Muse” (registered trademark) and “MindWave” are names of consumer EEG systems.

In FIG. 9, sites indicated by hollow circles are measurement points used only by the medically approved EEG systems. Sites AF7, Ap1, AF8, A1, and A2, on the other hand, are measurement points used only by Muse, which is a consumer EEG system. Fp1 is a measurement point used by all the four EEG systems. That is, Fp1 is a measurement point of MindWave. The measurement points A1 and A2 are located between the auricles and the temples, not in the external auditory meatuses.

Details of the thesis are not described here, but brain waves of five healthy subjects at rest are measured on two separate days. In this experiment, Fp1 on the forehead is used as a common measurement point, and brain wave patterns and power spectrum densities in an open eye state and a closed eye state are compared with each other. Evaluation in the thesis corresponds to evaluation of output a waves in the closed eye state.

In addition, in a “Conclusion” section of the thesis, it is described that power spectra measured by MindWave at Fp1 are substantially the same as with B-Alert and Enobio, which are the medically approved EEG systems, including results of reproduction tests, and peaks of a waves have been detected. It is also described that brain waves measured by MindWave include, as noise, blinking and movement in the open eye state. The thesis pointed out, as a reason why the reliability of Muse is low, the possibility of an effect of artifacts.

### Comparison between Results of Measurement with Earphone Terminal 1 and Results of Measurement with MindWave

Results of an experiment in which brain waves have been measured with the subjects wearing the earphone terminal 1 (refer to FIGS. 2A and 2B) or MindWave will be described hereinafter.

As illustrated in FIG. 8, the earphone terminal 1 uses the exterior auditory meatuses as measurement points, and MindWave uses the forehead 20A as a measurement point.

In the experiment conducted by the present applicant, there are 58 subjects. Three attention enhancement tests and three meditation enhancement tests on the same day are designed for each subject, and a waves observed in the closed eye state are measured.

Although the actual number of subjects is 83, an effect of artifacts in the open eye state is excessive in results of measurement performed on 25 subjects, and these results are excluded.

In each attention enhancement test, the subjects are instructed to keep looking at a tip of a pen 150 mm away for 30 seconds with their eyes open. This test creates a concentrating state to suppress appearance of α waves and increase β waves.

In each meditation enhancement test, the subjects are instructed to meditate for 30 seconds with their eyes closed. This test corresponds to evaluation of output α waves in the closed eye state. In other words, this test aims to detect the percentage of increase in α waves in a relaxed state.

In the experiment, the meditation enhancement test is conducted after the attention enhancement test, and output α waves are evaluated.

When output α waves are evaluated, a subject is usually instructed to keep his/her eyes open for 30 seconds and then keep his/her eyes closed for 30 seconds. This process is repeated twice, and an increase in α waves in the closed eye state is detected.

In the present experiment, however, the number of sets is increased in order to collect a large amount of data at once.

First, a reason why the meditation enhancement tests are conducted and a method used in the evaluation of output α waves in the closed eye state will be described.

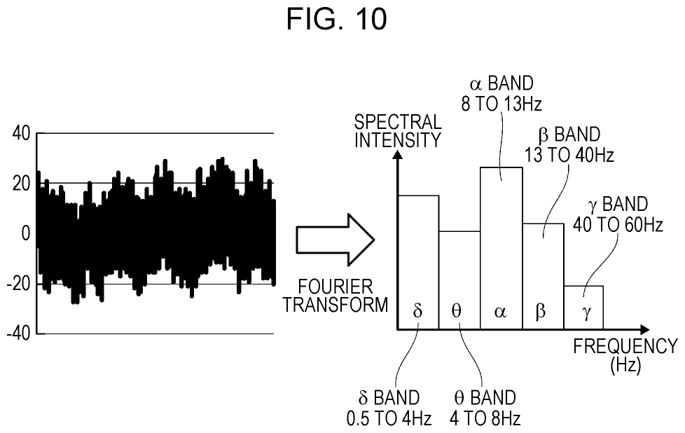

FIG. 10 is a diagram illustrating the evaluation of output α waves. As illustrated in FIG. 10, raw data regarding brain waves can be roughly classified into δ waves, θ waves, α waves, β waves, and γ waves.

It is considered that the reproducibility of brain waves based on human motion is low and evaluation of the reproducibility of acquisition performance based on clinical data is difficult. α waves, however, tend to remain constant regardless of whether a person's eyes are open or closed.

Every type of brain wave tends to be observed in the open eye state, but every type of brain wave other than α waves tends to attenuate in the closed eye state. That is, a waves can be relatively easily observed without being affected even in the closed eye state.

On the basis of this characteristic, raw data regarding brain waves in the experiment is subjected to a Fourier transform, and a spectral intensity Sn in a frequency band corresponding to each type of brain wave is determined as a feature value.

In the experiment, an α wave intensity ratio Ta is defined as a ratio (=Sα/ΣSn) of a spectral intensity Sα in an α band to the sum of spectral intensities in all the frequency bands (i.e., ΣSn), and whether the α wave intensity ratio Tα has increased in the closed eye state is determined.

If an increase in the α wave intensity ratio Tα is observed, it is proved that brain waves have been measured.

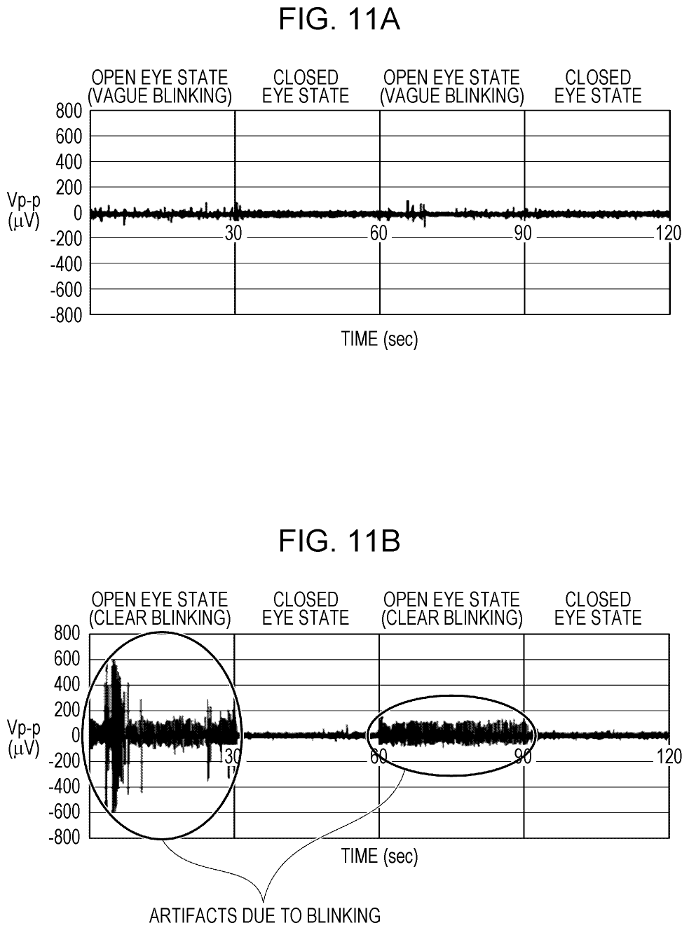

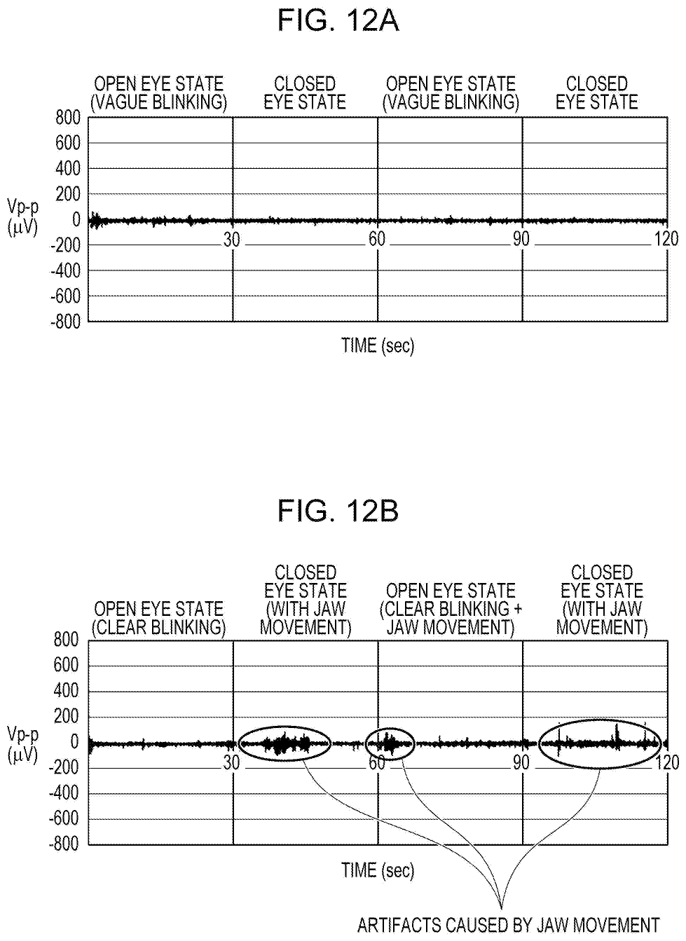

The comparison between the results of the measurement performed with the earphone terminal 1 and the results of the measurement performed with MindWave will be described with reference to FIGS. 11A to 12B.

FIGS. 11A and 11B are diagrams illustrating the results of the measurement performed with MindWave. FIG. 11A illustrates a result of measurement at a time when the open eye state and the closed eye state are alternated twice with subjects whose blinking is vague, and FIG. 11B illustrates a result of measurement at a time when the open eye state and the closed eye state are alternated twice with subjects whose blinking is clear.

FIGS. 12A and 12B are diagrams illustrating the results of the measurement performed with the earphone terminal 1 (refer to FIGS. 2A and 2B) used in the present exemplary embodiment. FIG. 12A illustrates a result of measurement at a time when the open eye state and the closed eye state are alternated twice with subjects whose blinking is vague, and FIG. 12B illustrates a result of measurement at a time when the open eye state and the closed eye state are alternated twice with subjects whose blinking is clear and who are asked to move the jaw.

With the subject whose blinking is vague, the result of the measurement performed with the earphone terminal 1 and the result of the measurement performed with MindWave are closely similar to each other.

With the subjects whose blinking is clear, however, artifacts caused by the blinking are evident in the results of the measurement performed with MindWave. This is probably because the forehead, which is used for measurement by MindWave, is close to the eyes and blinking in the open eye state tends to be detected as major artifacts. This has also been pointed out in the above-described thesis by Elena Ratti, et al.

Most of the artifacts due to blinking are observed in the δ band. When there are major artifacts as in FIG. 11B, however, an increase in α waves might be erroneously detected. This is because, as a result of an increase in the sum of the spectral intensities in all the frequency bands in the open eye state, the α wave intensity ratio Tα in the open eye state decreases, and the α wave intensity ratio Tα in the closed eye state looks relatively large. This is why the number of subjects has been reduced.

The artifacts caused by blinking include not only potential variations derived from a living body due to movement of the eyelids but also potential variations derived from brain waves caused when the subjects intend to move their eyelids.

In the result of the measurement performed with the earphone terminal 1 (refer to FIGS. 2A and 2B) according to the present exemplary embodiment, on the other hand, artifacts due to blinking have not been detected in a period of 0 to 30 seconds.

It has been confirmed, however, that artifacts due to a movement of the jaw for swallowing saliva are detected regardless of whether the subjects' eyes are open or closed. Most of the artifacts due to the movement of the jaw for swallowing saliva have been observed in the θ band.

The spectral intensity of the artifacts caused by swallowing of saliva, however, is much lower than that of the artifacts corresponding to blinking detected by MindWave. The spectral intensity of the artifacts caused by swallowing of saliva, therefore, has not affected an increase in α waves unlike in the case of MindWave.

The artifacts caused by swallowing of saliva, too, include not only potential variations derived from a living body due to movement of the muscles in the jaw but also potential variations derived from brain waves caused when the subjects intend to move the muscles in their jaws.

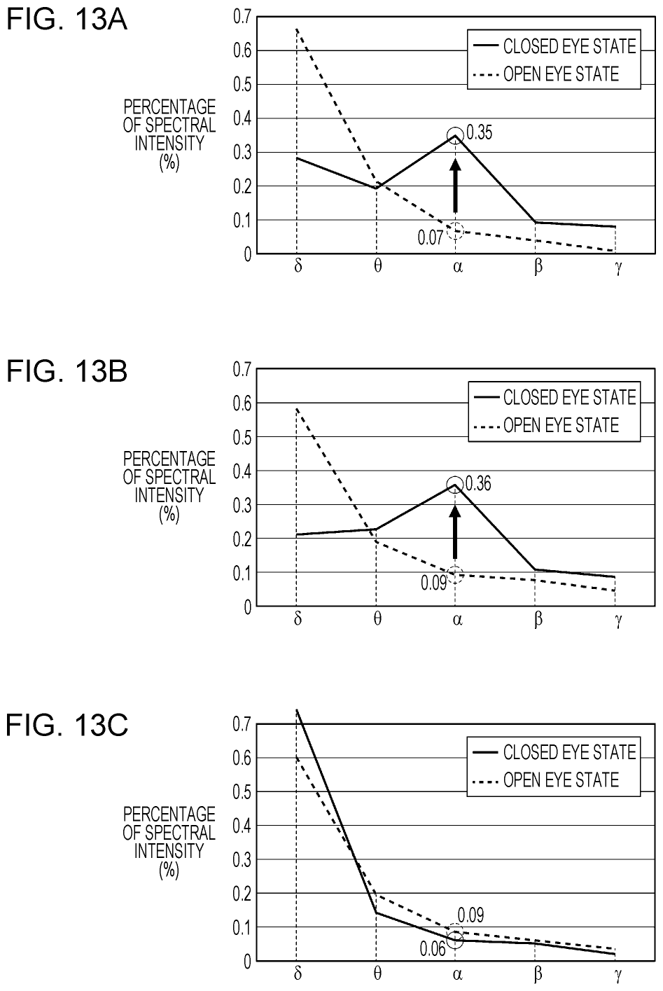

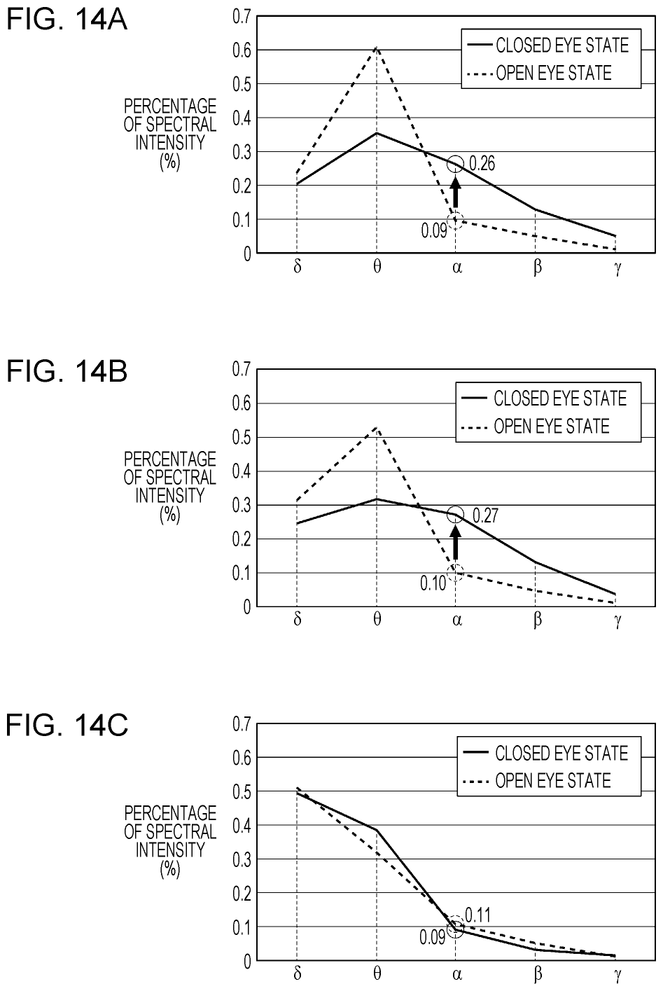

Next, an increase in α waves observed in the result of the measurement performed with the earphone terminal 1 and an increase in α waves observed in the result of measurement performed with MindWave will be described with reference to FIGS. 13A to FIG. 14C.

FIGS. 13A to 13C are diagrams illustrating other results of the measurement performed with MindWave. FIG. 13A illustrates changes in the percentage of spectral intensity in each frequency band at a time when the subjects have entered the closed eye state from the open eye state with clear blinking. FIG. 13B illustrates changes in the percentage of spectral intensity in each frequency band at a time when the subjects have entered the closed eye state from the open eye state with vague blinking. FIG. 13C illustrates a case where a waves do not increase.

FIGS. 14A to 14C are diagrams illustrating other results of the measurement performed with the earphone terminal 1 (refer to FIGS. 2A and 2B) used in the present exemplary embodiment. FIG. 14A illustrates changes in the percentage of spectral intensity in each frequency band at a time when the subjects have entered the closed eye state from the open eye state with clear blinking. FIG. 14B illustrates changes in the percentage of spectral intensity in each frequency band at a time when the subjects have entered the closed eye state from the open eye state with vague blinking. FIG. 14C illustrates a case where a waves do not increase.

Vertical axes in FIGS. 13A to 14C represent the percentage of spectral intensity, and horizontal axes represent the frequency bands. Subjects corresponding to FIG. 13A are the same as those corresponding to FIG. 14A. Similarly, subjects corresponding to FIG. 13B are the same as those corresponding to FIG. 14B, and subjects corresponding to FIG. 13C are the same as those corresponding to FIG. 14C.

The distribution of the spectral intensity of MindWave (refer to FIGS. 13A to 13C) and the distribution of the spectral intensity of the earphone terminal 1 (refer to FIGS. 14A to 14C) are different from each other in low frequency bands of δ waves to θ waves, but the same in the α band and higher.

In the experiment, the number of subjects with whom an increase in α waves has been observed with both MindWave and the earphone terminal 1 is 46, which is slightly less than 80% of the total number of subjects, namely 58.

The number of subjects with whom an increase in a waves has been observed only with the earphone terminal 1 is seven. In other words, an increase in α waves has been observed with 53 subjects in the case of the earphone terminal 1. That is, in the case of the earphone terminal 1, an increase in α waves has been observed with slightly more than 90% of the total number of subjects.

The number of subjects with whom an increase in α waves has been observed with neither MindWave nor the earphone terminal 1 is five. Waveforms illustrated in FIGS. 13C and 14C indicate results of measurement performed on the five subjects.

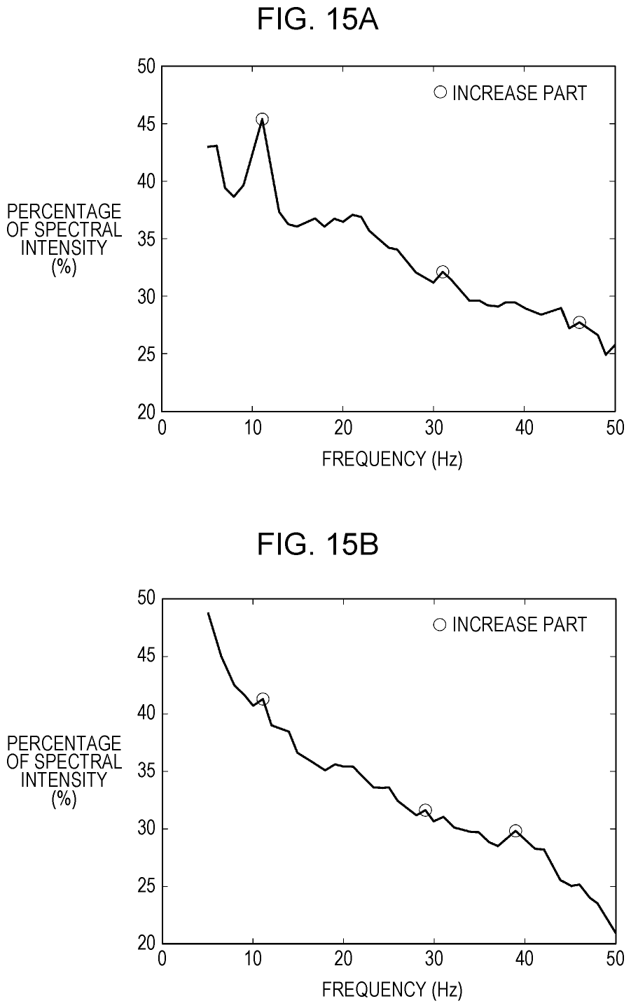

FIGS. 15A and 15B are diagrams illustrating an example of presentation of parts in which spectral intensity has increased. FIG. 15A illustrates a result of the measurement performed with MindWave, and FIG. 15B illustrates a result of the measurement performed with the earphone terminal 1 (refer to FIGS. 2A and 2B) used in the present exemplary embodiment. Vertical axes represent the percentage of spectral intensity, and horizontal axes represent frequency.

In FIGS. 15A and 15B, unlike in FIGS. 13A to 14C, actual frequency is used for the horizontal axes. In the above-described thesis by Elena Ratti, et al., horizontal axes represent actual frequency to describe an increase in a waves. The parts in which spectral frequency has increased are indicated by hollow circles in FIGS. 15A and 15B.

As illustrated in FIGS. 15A and 15B, in either measurement method, the percentage of spectral intensity decreases as the frequency increases. This holds true in the thesis by Elena Ratti, et al.

It has thus been confirmed that the earphone terminal 1 used in the present exemplary embodiment, which measures brain waves at the exterior auditory meatuses, has measurement capability equivalent to that of MindWave.

### Second Exemplary Embodiment

In a second exemplary embodiment, a process performed when a target who is not concentrating receives a telephone call.

In the present exemplary embodiment, too, the earphone terminal 1 described in the first exemplary embodiment is used. Differences in the process are caused by a program executed by the MPU 16 (refer to FIG. 3).

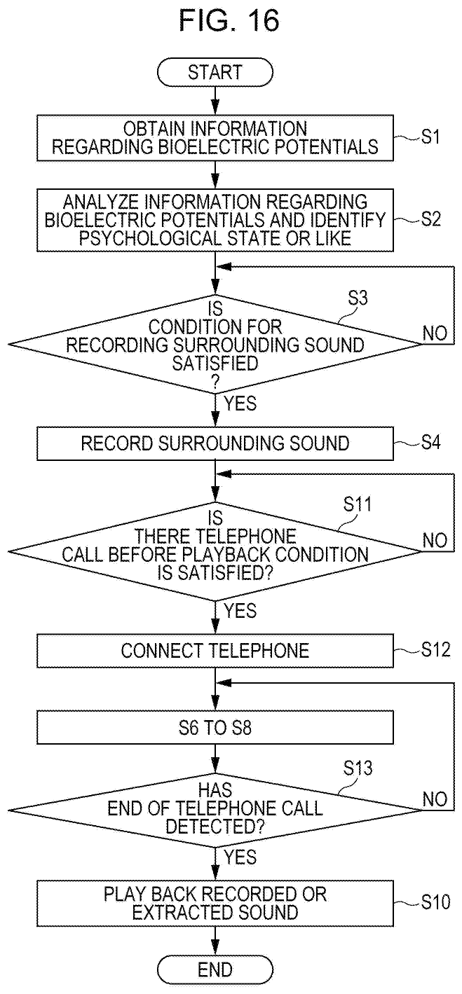

FIG. 16 is a flowchart illustrating an example of the process performed by the earphone terminal 1 used in the second exemplary embodiment. In FIG. 16, the same steps are given the same reference numerals as in FIG. 5.

In the present exemplary embodiment, too, the earphone terminal 1 obtains information regarding bioelectric potentials (S1). The earphone terminal 1 then analyzes the information regarding bioelectric potentials and identifies a psychological state or the like (S2).

The earphone terminal 1 determines whether the condition for recording surrounding sound is satisfied (S3). While a result of S3 is negative, the earphone terminal 1 repeats S3.

If the result of S3 is positive, on the other hand, the earphone terminal 1 records the surrounding sound (S4).

The process so far is the same as in the first exemplary embodiment.

Next, the earphone terminal 1 determines whether there is a telephone call before the playback condition is satisfied (S11). In the present exemplary embodiment, the earphone terminal 1 repeats S11 while a result of S11 is negative. S5 to S10 described in the first exemplary embodiment, however, are repeated even while the result of S11 is negative. It is therefore possible that playback of the recorded surrounding sound starts before the result of S11 becomes positive.

It is assumed here that the result of S11 is positive. That is, it is assumed here that there is a telephone call before the playback condition is satisfied. In this case, the earphone terminal 1 connects the telephone call to the earphone terminal 1 (S12). In the present exemplary embodiment, the earphone terminal 1 is associated with the wearer's telephone, smartphone, or the like.

When the telephone call is connected to the earphone terminal 1, the wearer begins to talk with a person who has made the telephone call. When the wearer begins to talk with the person, the wearer who might have been bored usually concentrates. In the first exemplary embodiment, this change is detected as an event for ending recording of surrounding sound, and playback of audio data recorded so far starts.

In the present exemplary embodiment, however, the wearer is talking with the person and does not need playback of surrounding sound. The earphone terminal 1 according to the present exemplary embodiment, therefore, keeps recording surrounding sound while the wearer is talking on the phone even after the psychological state or the like of the wearer changes. S6 to S8 described in the first exemplary embodiment, therefore, are repeatedly performed even while the wearer is talking on the phone.

The earphone terminal 1 then determines whether an end of the telephone call has been detected (S13). An end of a telephone call can be detected as a notification from the wearer's telephone or smartphone. While a result of S13 is negative, S6 to S8 are repeated for surrounding sound that is being recorded.

If the result of S13 is positive, the earphone terminal 1 plays back the recorded or extracted sound (S10). That is, surrounding sound during the telephone call is played back.

In the present exemplary embodiment, a case where the result of S3 becomes positive is the same as in the first exemplary embodiment, but it is not appropriate to answer a telephone call if the wearer is irritated. The result of S3, therefore, may become positive only in predetermined states, such as when it is determined that the wearer is bored.

### Other Exemplary Embodiments

Although some exemplary embodiments of the present disclosure have been described, the technical scope of the present disclosure is not limited to that of the above exemplary embodiments. It is obvious from the claims that the technical scope of the present disclosure also includes modes obtained by modifying or improving the above exemplary embodiments in various ways.

For example, although the earphone terminal 1 (refer to FIGS. 2A and 2B) performs all the above-described processes in the above-described exemplary embodiments, an external apparatus may perform part or the entirety of the processes, instead. In this case, the external apparatus, or a combination of the external apparatus and the earphone terminal 1, is an example of the information processing apparatus.

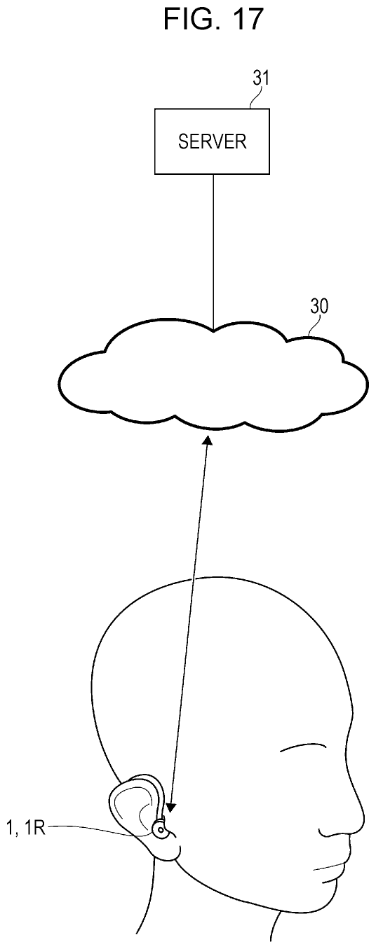

FIG. 17 is a diagram illustrating a case where the external apparatus is a server 31 on the Internet 30. In FIG. 17, the earphone terminal 1 functions as a device for uploading information regarding brain waves measured in the wearer to the server 31 and receiving a result of a process.

In addition, although brain waves are an example of the information regarding bioelectric potentials that can be measured by the earphone terminal 1 (refer to FIGS. 1A and 1B) in the above-described exemplary embodiments, myoelectric potentials, heartbeats, heart potentials, pulsation, or pulse waves may be used, instead.

Although brain waves are measured with the earphone terminal 1 inserted into the exterior auditory meatuses in the above exemplary embodiments, an earphone terminal 1 to be inserted into one of the exterior auditory meatus may be used, instead.

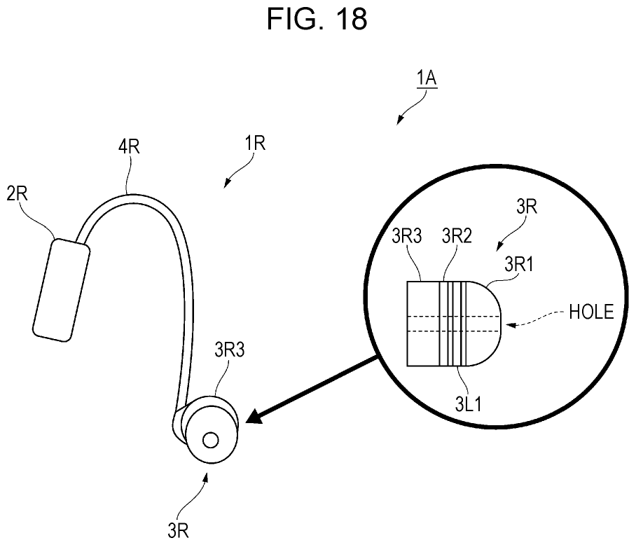

FIG. 18 is a diagram illustrating an example of the appearance of an earphone terminal 1A to be inserted into one of the ears. In FIG. 18, the same components as in FIGS. 2A and 2B are given the same reference numerals. The earphone terminal 1A illustrated in FIG. 18 includes the module 1R to be attached to the right ear as a basic component. In FIG. 18, the module body 2R includes the lithium-ion battery 17 (refer to FIG. 3).

In the earphone terminal 1A illustrated in FIG. 18, three electrodes 3R1, 3L1, and 3R2 are provided at a tip of the vibration unit 3R to be attached to the ear. The dome-shaped electrode 3R1 and the ring-shaped electrode 3L1, and the ring-shaped electrode 3L1 and the ring-shaped electrode 3R2, are electrically isolated from each other by an insulator.

Although an example in which electrodes for measuring potential variations caused by brain waves and the like are provided for the earphone terminal 1 has been described in the above-described exemplary embodiments, the electrodes may be provided for another article or device, instead. Some specific examples will be described hereinafter.

For example, the electrodes for measuring potential variations caused by brain waves and the like may be provided for headphones that cover the auricles. In the case of headphones, the electrodes are provided at parts of earpads that come into contact with the head. At this time, the electrodes are arranged at positions where there is little hair and the electrodes can come into direct contact with the skin.

The article that comes into contact with an auricle may be a fashion accessory such as an earring or a spectacle-shaped device. These are examples of a wearable device.

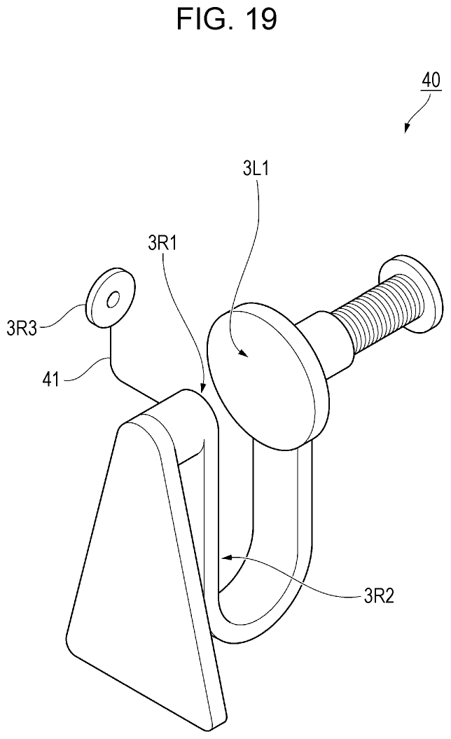

FIG. 19 is a diagram illustrating an example of an earring 40 for which the electrodes for measuring brain waves are provided. The earring 40 illustrated in FIG. 19 includes the electrode 3R1 that comes into contact with an earlobe on a front side of the ear, on which an ornament is to be attached, the electrode 3L1 that comes into contact with the earlobe on a back side of the ear, and the electrode 3R2 that comes into contact with the earlobe at some position of a U-shaped body thereof. These electrodes are electrically isolated from one another by an insulator, which is not illustrated. A battery for supplying power necessary for operation and a communication module such as Bluetooth are incorporated into the ornament, the U-shaped body, a screw for moving a plate-like member on which the electrode 3L1 is arranged, or the like.

The cartilage conduction vibrator 3R3 is connected to the earring 40 by cable 41. In this case, the cartilage conduction vibrator 3R3 is separately attached to the ear.

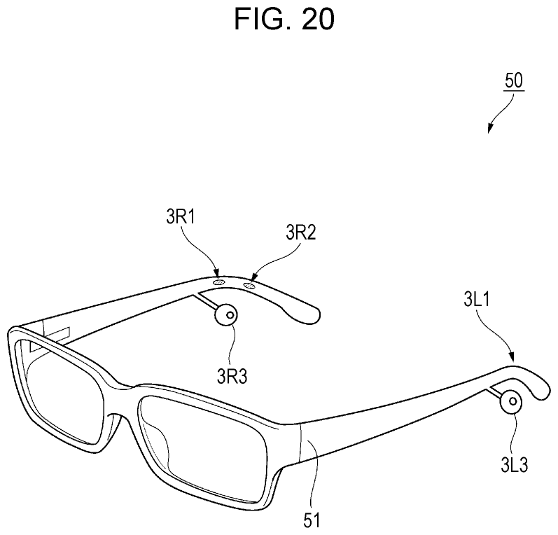

FIG. 20 is a diagram illustrating an example of spectacles 50 for which the electrodes for measuring brain waves are provided. In the spectacles 50 illustrated in FIG. 20, the electrodes 3R1 and 3R2 are provided on a tip of a right temple (hereinafter referred to as a “temple tip”) 51, and the electrode 3L1 is provided on a tip of a left temple 51. These electrodes are electrically isolated from one another by an insulator, which is not illustrated. A battery for supplying power necessary for operation and a communication module such as Bluetooth are incorporated into a temple or a temple tip. In FIG. 20, the cartilage conduction vibrators 3R3 and 3L3 are connected to the temple tips, respectively.

The electrodes for measuring brain waves may be incorporated into smart glasses or a headset called “head-mounted display” that displays information, instead. The electrodes may be mounted on a headset having a function of detecting a surrounding environment of the user and displaying an image assimilating to the surrounding environment, instead.

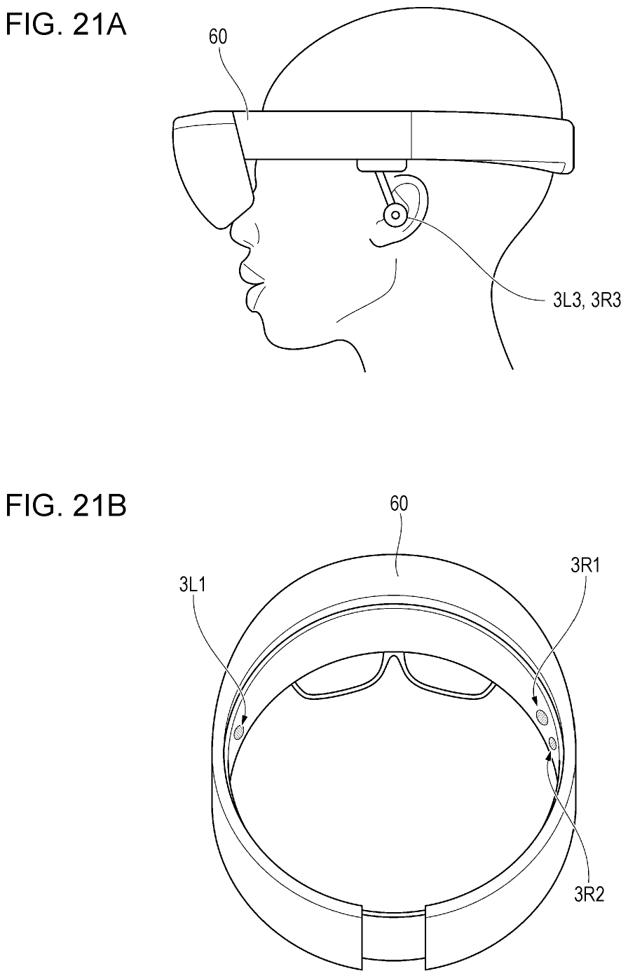

FIGS. 21A and 21B are diagrams illustrating an example in which the electrodes for measuring brain waves are provided for a headset 60 having a function of displaying an image assimilating to the surrounding environment of the user. The headset 60 illustrated in FIGS. 21A and 21B has a configuration in which the electrodes for measuring brain waves are provided for hololens (registered trademark) manufactured by Microsoft (registered trademark) Corporation. A virtual environment experienced by the user wearing the headset 60 is called “augmented reality” or “mixed reality”.

In the headset 60 illustrated in FIGS. 21A and 21B, the electrodes 3R1, 3R2, and 3L1 are arranged in parts of a ring-shaped member that come into contact with the ears, the ring-shaped member being attached to the head. In the case of the headset 60 illustrated in FIGS. 21A and 21B, the electrodes 3R1 and 3R2 are arranged on a side of the right ear, and the electrode 3L1 is arranged on a side of the left ear. When a function of tracking a line of sight provided for the headset 60 is used, an object or a person that the wearer is looking at can be easily associated with the psychological state or the like of the wearer. The cartilage conduction vibrator 3R3 to be attached to the right ear and the cartilage conduction vibrator 3L3 to be attached to the left ear are incorporated into the headset 60.

Although a case where biological information including brain waves is obtained using electrodes in contact with the user's ears has been described in the above exemplary embodiments, positions at which the biological information including brain waves is obtained is not limited to the ears. For example, the electrodes may be provided on the forehead or another part on the head, instead.

In the case of the headset 60 (refer to FIGS. 21A and 21B), for example, the electrodes may be provided at some positions on the ring-shaped member attached to the head.

Although biological information including brain waves is obtained using the electrodes in contact with the user's head including the ears in the above exemplary embodiments, brain activity may be measured on the basis of changes in the amount of blood flow, instead.

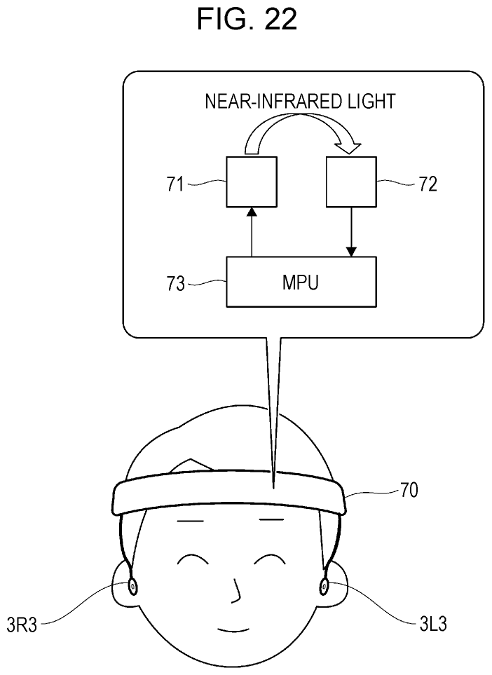

FIG. 22 is a diagram illustrating an example of a headset 70 that measures changes in the amount of blood flow caused by brain activity using near-infrared light. The headset 70 includes a ring-shaped body attached to the head. Inside the body, one or more measurement units each including a probe 71 that radiates near-infrared light onto the scalp and a detection probe 72 that receives reflected light are provided. An MPU 73 controls the radiation of near-infrared light by the probes 71 and detects features of the user's brain waves by processing signals output from the detection probes 72.

Alternatively, a magnetoencephalograph (MEG) may be used to obtain biological information including brain waves. A tunnel magnetoresistance (TMR) sensor, for example, is used to measure magnetic fields caused by electrical activity of nerve cells of the brain.

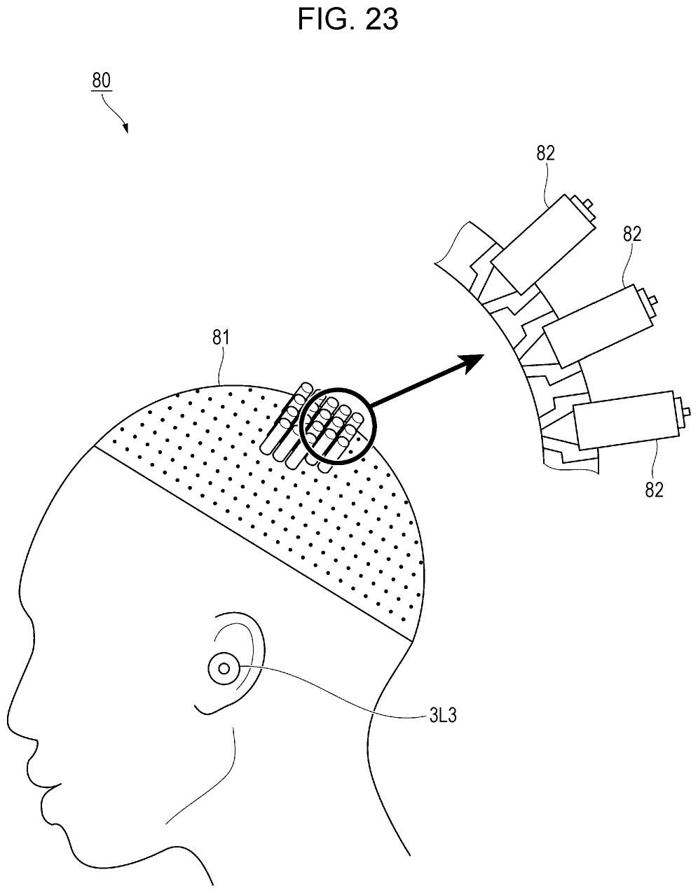

FIG. 23 is a diagram illustrating an example of an MEG 80. The MEG 80 illustrated in FIG. 23 has a structure in which TMR sensors 82 are arranged on a cap 81 attached to the head. Outputs of the TMR sensors 82 are input to an MPU, which is not illustrated, and a magnetoencephalogram is generated. In this case, the distribution of magnetic fields in the magnetoencephalogram is used as features of the user's brain waves. FIG. 23 also illustrates the cartilage conduction vibrator 3L3 attached to the ear.

Although cartilage conduction is employed on the assumption that sound is to be transmitted to the wearer without being noticed by others in the above-described exemplary embodiments, bone conduction may be employed, instead. When bone conduction is employed, bone conduction vibrators are disposed at the wearer's temples. Alternatively, earphones including diaphragms that output sound may be used instead of cartilage conduction or bone conduction.

The Bluetooth module 14 of the earphone terminal 1 described in the above-described exemplary embodiments may conform to Bluetooth low-energy (LE) Audio. Bluetooth LE Audio is disclosed, for example, in “https://www.bluetooth.com/learn-about-bluetooth/bluetooth-technology/le-audio/” and the like. When the Bluetooth module 14 conforms to the standard, an emergency broadcast or the like received while the earphone terminal 1 was being used can be superimposed upon sound that is being played back. The outputting is an example of use of a broadcast function of Bluetooth LE Audio or a function of simultaneously connecting plural devices to one device. The earphone terminal 1 corresponds to the plural devices.

In the embodiments above, the term “MPU” refers to hardware in a broad sense. Examples of the MPU include general processors (e.g., CPU: Central Processing Unit) and dedicated processors (e.g., GPU: Graphics Processing Unit, ASIC: Application-Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to, if information regarding a psychological state or a feeling of a target satisfies a predetermined first condition, control outputting of sound around the target collected in a period in which the predetermined first condition is satisfied.

2. The information processing apparatus according to claim 1,
wherein the predetermined first condition is a state in which the target is not concentrating.

3. The information processing apparatus according to claim 2,
wherein the predetermined first condition is that a level of θ waves or a level of δ waves measured in the target is higher than a predetermined threshold.

4. The information processing apparatus according to claim 2,
wherein the processor is configured to, if a telephone call is detected, enables the telephone call.

5. The information processing apparatus according to claim 1,
wherein the predetermined first condition is a state in which the target is aroused.

6. The information processing apparatus according to claim 5,
wherein the predetermined first condition is a state in which a level of δ waves or a level of β waves measured in the target is higher than a predetermined threshold.

7. The information processing apparatus according to claim 1,
wherein the processor is configured to, if a predetermined second condition is satisfied, output the sound around the target collected in the period in which the first condition is satisfied.

8. The information processing apparatus according to claim 7,
wherein the predetermined second condition is that the predetermined first condition is not satisfied.

9. The information processing apparatus according to claim 7,
wherein the predetermined second condition is detection of an instruction, given by the target, to output the sound.

10. The information processing apparatus according to claim 7,
wherein the predetermined second condition is set for each target.

11. The information processing apparatus according to claim 7,
wherein the predetermined second condition is detection of a change in an environment of the target.

12. The information processing apparatus according to claim 11,
wherein the change in the environment is detection of an end of a telephone call.

13. The information processing apparatus according to claim 11,
wherein the change in the environment is detection of an end of a meeting attended by the target.

14. The information processing apparatus according to claim 1,
wherein the processor is configured to play back, faster than a normal speed, the sound around the target collected in the period in which the first condition is satisfied.

15. The information processing apparatus according to claim 1,
wherein the processor is configured to output a summary of the sound around the target collected in the period in which the first condition is satisfied.

16. The information processing apparatus according to claim 1,
wherein the processor is configured to, if the sound around the target collected in the period in which the first condition is satisfied satisfies a predetermined third condition, output the sound that satisfies the predetermined third condition.

17. The information processing apparatus according to claim 16,
wherein the predetermined third condition is a talk given by a certain speaker.

18. The information processing apparatus according to claim 16,
wherein the predetermined third condition is set for each target.

19. The information processing apparatus according to claim 17,
wherein the predetermined third condition is set for each target.

20. A non-transitory computer readable medium storing a program causing a computer to execute a process comprising:
controlling, if information regarding a psychological state or a feeling of a target satisfies a predetermined first condition, outputting of sound around the target collected in a period in which the predetermined first condition is satisfied.
